(19) United States
(12) Patent Application Publication
(10) Pub. No.: US 2014/0011829 A1
(43) Pub. Date: Jan. 9, 2014

(54) Artemisinin-Based Combination Therapy For Treating Viral Mediated Disease

(71) Applicants: Robert Lewis Steele, Palm Beach Gardens, FL (US); Anthony Fedele Musso, West Palm Beach, FL (US)

(72) Inventors: Robert Lewis Steele, Palm Beach Gardens, FL (US); Anthony Fedele Musso, West Palm Beach, FL (US)

(21) Appl. No.: 13/542,702

(22) Filed: Jul. 6, 2012

(51) Int. Cl. A61K 31/4375; A61P 31/14

(52) U.S. Cl. 514/280

(57) ABSTRACT

The present invention describes a method of treating individuals suffering from microbial infections, including a viral infection such as Dengue Fever, using an improved Artemisinin Combination Therapy (ACT), known as Tri-ACT. The improved ACT therapy includes administering a combination of three drugs. In one embodiment of the present invention, the method includes administering to an individual a first composition comprising a therapeutically effective amount of an artemether spray sublingually. The individual is then administered a second composition, a therapeutically effective amount of artesunate. A third composition, an effective amount of berberine, or its pharmaceutically acceptable derivatives or salts is then administered to the individual.

### CROSS REFERENCE TO RELATED APPLICATIONS

The present invention is related to U.S. patent application Ser. No. ______, entitled “ARTEMISININ-BASED COMBINATION THERAPY FOR A PARASETIC MEDIATED DISEASE” filed on ______. (Attorney Docket No. 4254U.002)

### FIELD OF THE INVENTION

The present invention relates to microbial therapies, and particularly to a method of treating individuals suffering from a viral related disease using an improved Artemisinin based therapy, and more particularly to a method of treating an individual suffering from Dengue Fever using an improved Artemisinin Combination Therapy (ACT).

### BACKGROUND OF THE INVENTION

Dengue Fever is a fever spread by several species of mosquitoes within the Aedes species, most commonly by Aedes aegypti. It is caused by one of four similar RNA viruses of the family Flaviviridae, DEN-1, DEN-2, Den-3, and DEN-4. All four serotypes can result in full blown Dengue fever. Infection with a serotype of Dengue Fever may produce a spectrum of clinical disease from non-specific viral syndrome to severe fatal hemorrhagic disease. Symptoms of Dengue fever include sudden high fever, often in the range of 104-105 degrees Fahrenheit, about 4-7 days after the bite. A characteristic body rash, similar to the skin rash seen in measles, appears several days after the fever begins. In some cases, the disease progresses to dengue hemorrhagic fever, resulting in bleeding, low blood platelet levels, plasma leakage, and dengue shock syndrome.

Dengue Fever is the second most important infectious tropical disease after malaria, as more than one-third of the world's population live in areas at risk of transmission. There are estimated to be 200 million individuals infected with the Dengue virus every year, with 10,000,000 patients hospitalized for hemorrhagic dengue fever, with about 5% of such cases resulting in death. Dengue Fever virus infections are endemic in more than 100 tropical countries, including countries in Asia, the Pacific, Africa, Latin America and the Caribbean. While such places tend to have unplanned and uncontrolled urbanization, as well as high poverty, an increase in air travel, lack of effective mosquito control, and military deployment into risk regions can result in outbreaks in more developed countries. In 2009, Dengue infection was reported in the United States by a Florida resident who had not travelled outside the U.S.

There are no known treatments or vaccines to prevent infection with Dengue virus. In fact, the most effective measure to prevent the disease is to avoid mosquito bites. While protective clothing and mosquito irradiation programs theoretically provide protection, what is needed in the art is an easy to administer method of treating individuals suffering Dengue Fever.

### SUMMARY OF THE INVENTION

The present invention describes a therapy for individuals suffering from a viral infection based on Artemisinin Combination Therapy (ACT). In contrast to most ACT therapies which utilize a combination dual drug therapy, the present invention describes a method which uses a combination of three drugs. In one embodiment of the present invention, the method includes administering to an individual a first composition. The first composition comprises of a therapeutically effective amount of an artemisinin derivate or its salt, such as an artemether spray delivered sublingually. The individual is then administered a second composition. The second composition comprises of a therapeutically effective amount of a second artemisinin derivate or its' salt. The second artemisinin derivate differs from the first composition and is preferably artesunate. A third anti-microbial composition is administered to the individual. The third composition comprises an effective amount of berberine, or its pharmaceutically acceptable derivatives or salts. The second and third compositions are administered to the individual for additional periods, such as for two or three days.

In an alternative embodiment, the treatment is administered to an individual suffering from a disease transmitted by an arthropod, such as a mosquito or tick.

In an alternative embodiment, the treatment is administered to an individual suffering from a disease mediated by a virus of the Flaviviridae family of viruses.

In an alternative embodiment, the treatment is administered to an individual suffering from a disease mediated by a virus similar to viruses in the Flaviviridae family of viruses.

In an alternative embodiment, the treatment is administered to an individual suffering from a disease mediated by a virus causing hemorrhagic fever.

In an alternative embodiment, the treatment is administered to an individual suffering from a disease mediated by a Dengue Fever virus.

As used herein, the term “Flaviviridae family of viruses” includes about 70 members which have linear, single-stranded RNA genomes of positive polarity. The family includes the Genus Flavivirus, Genus Hepacivirus, Genus Hepatitis G Virus, and Genus Pestivirus. Major diseases caused by the Flaviviridae family include: Dengue fever, Japanese encephalitis, Kyasanur Forest disease, Murray Valley encephalitis, St. Louis encephalitis, Tick-borne encephalitis, West Nile encephalitis, Yellow fever, and Hepatitis C Virus Infection.

As used herein, the term “pharmaceutically acceptable excipient” or “pharmaceutically acceptable carrier,” generally refers to organic or inorganic materials, non-toxic, inert solid, semi-solid or liquid filler, diluent, encapsulating material or formulation auxiliary of any type which cannot react with active ingredients. Excipients include but are not limited to sugars, such as lactose, glucose and sucrose; starches such as corn starch and potato starch; cellulose and its derivatives such as sodium carboxymethylcellulose, ethylcellulose, and cellulose acetate; powered tragacanth; malt; gelatin; talc; stearic acids; magnesium stearate; calcium sulfate; cocoa butter and suppository waxes; vegetable oils, such as peanut oil, cotton seed oil, sesame oil, olive oil, corn oil and oil of theobroma; esters such as but not limited to ethyl oleate and ethyl laurate; polyols such as propylene glycol, glycerine, sorbitol, mannitol, and polyethylene glycol; agar; alginic acids; buffering agents such as but not limited to, magnesium hydroxide and aluminum hydroxide; pyrogen-free water; isotonic saline; and phosphate buffer solution; skim milk powder; as well as other non-toxic compatible substances used in pharmaceutical formulations. Wetting agents and lubricants such as sodium lauryl sulfate and magnesium stearate, as well as coloring agents, flavoring agents, sweetening agents, lubricants, releasing agents, perfuming agents, carriers, tabletting agents, stabilizers, antioxidants and preservatives can also be present.

As use herein, “Pharmaceutically-acceptable salt” refers to salts which retain the biological effectiveness and properties of compounds which are not biologically or otherwise undesirable. Pharmaceutically-acceptable salts refer to pharmaceutically-acceptable salts of the compounds, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, the term “therapeutically effective amount” generally refers to an amount of an agent, for example the amount of a compound as an active ingredient, that is sufficient to effect treatment as defined herein when administered to an individual, such as a mammal, preferably a human in need of such treatment. A therapeutically effective amount of a compound, salt, analog, or derivative of the present invention will depend on a number of factors including, for example, the age and weight of the subject, the precise condition requiring treatment and its severity, the nature of the formulation, and the route of administration, and will ultimately be at the discretion of the attendant physician.

As used herein, the term “treat”, “treating” or “treatment” refers to the administration of therapy to a subject, particularly a mammal, more particularly a human, who already manifests at least one symptom of a disease to obtain a desired pharmacological and physiological effect. Such a subject includes an individual who is diagnosed as having a disease. The term may also include preventing the disease, i.e. causing the clinical symptoms of the disease not to develop in a mammal that may be exposed to or predisposed to the disease but does not yet experience or display symptoms of the disease, inhibiting the disease, i.e., arresting or reducing the development of the disease or its clinical symptoms, or relieving the disease, i.e., causing regression of the disease or its clinical symptoms.

As used herein, “virus causing hemorrhagic fever” describes RNA viruses such as those of Filoviridae, Arenaviridae, Bunyaviridae or Flaviviridae family, include but are not limited to ebola viruses, marburg viruses, flexal viruses, guanarito viruses, jumin viruses, lassa fever viruses, machupo viruses, sabia viruses, crimea-congo hemorrhagic fever viruses, rift valley fever viruses, hantaan viruses, dengue viruses, kyasanur forest disease viruses, omsk hemorrhagic fever viruses, and yellow fever viruses. In one aspect of the invention, the Hemorrhagic fever viruses are dengue viruses.

Accordingly, it is an objective of the present invention to provide an improved Artemisinin Combination Therapy to individuals in need thereof.

It is a further objective of the present invention to provide an improved Artemisinin-based Combination Therapy to individuals suffering from a viral disease.

It is a further objective of the present invention to provide an improved Artemisinin-based Combination Therapy to individuals suffering from a viral disease mediated by arthropods, such as a mosquito.

It is a still further objective of the present invention to provide an improved Artemisinin-based Combination Therapy to individuals suffering from a viral disease mediated by a virus of the family Flaviviridae.

It is a further objective of the present invention to provide an improved Artemisinin-based Combination Therapy incorporating a sublingual delivery step to individuals suffering from a viral disease.

It is yet another objective of the present invention to provide an improved Artemisinin-based Combination Therapy incorporating a sublingual delivery step to individuals suffering from a viral disease mediated by a virus of the family Flaviviridae.

It is a still further objective of the present invention to provide an improved Artemisinin-based Combination Therapy incorporating delivery of an Artemisinin derivative using a sublingual, spray-based delivery route of administration to individuals suffering from Dengue Fever.

It is yet another objective of the present invention to provide an improved Artemisinin-based Combination Therapy incorporating delivery of an Artemisinin derivative using a sublingual, spray-based delivery route of administration in combination with oral delivery of a second, independent artemisinin derivative and a berberine, or its derivatives, for individuals suffering from a viral disease.

It is a still further objective of the present invention to provide an improved artemisinin-based Combination Therapy incorporating delivery of an artemisinin derivative using a sublingual, spray-based delivery route of administration in combination with oral delivery of a second, independent artemisinin derivative and a berberine, or its derivatives, for individuals suffering from a disease mediated by an arthropod.

It is a further objective of the present invention to provide an improved Artemisinin-based Combination Therapy incorporating delivery of an artemisinin derivative using a sublingual, spray-based delivery route of administration in combination with oral delivery of a second, independent artemisinin derivative and a berberine, or its derivatives, for individuals suffering from a Dengue Fever.

It is yet another objective of the present invention to provide an improved Artemisinin-based Combination Therapy incorporating delivery of an Artemisinin derivative using a sublingual, spray-based delivery route of administration in combination with oral delivery of a second, independent artemisinin derivative and a berberine, or its derivatives, for individuals suffering from a disease mediated by an arthropod.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### DETAILED DESCRIPTION OF THE INVENTION

While the present invention is susceptible of embodiment in various forms, there is demonstrated and will hereinafter be described a presently preferred, albeit not limiting, embodiment with the understanding that the present disclosure is to be considered an exemplification of the present invention and is not intended to limit the invention to the specific embodiments illustrated.

The present invention describes a novel combination therapy for the treatment of microbial infections, such as viral infections. As an illustrative example, the present invention has been found to be useful in treating diseases mediated by arthropods and/or a viral infection from the Flaviviridae family, such as the viruses causing Dengue Fever. The combination therapy in accordance with the present invention is based on Artemisinin Combination Therapy (ACT). The present invention uses a tri-therapy modality, using a unique combination of three compositions: artemether, artesunate, and berberine.

Artemisinin and Artemisinin Derivatives

Artemisinin, having an IUPAC name of 3R,5aS,6R,8aS,9R,12S,12aR)-octahydro-3,6,9-trimethyl-3,12-epoxy-12H-pyrano[4,3-j]-1,2-benzodioxepin-10(3H)-one, and also known as Qinhaosu, is a natural product derived from the Chinese herb Artemisia annua known to have anti-viral activities, see Efferth et al. The Antiviral Activities of Artemisinin and Artesunate. Clin. Inf. Dis. 2008:47:804-11. It has a chemical structure of:

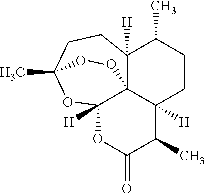 [SMILES: C[C@@H]1CC[C@H]2[C@@H](C)C(=O)O[C@@H]3O[C@]4(C)CC[C@@H]1[C@]32OO4]

Artemisinin is a sesquiterpene lactone containing an unusual peroxide bridge. This peroxide is believed to be responsible for the drug's mechanism of action. Few other natural compounds with such a peroxide bridge are known; see Artemisinin and a new generation of antimalarial drugs”. Education in Chemistry. July 2006. http://www.rsc.org/Education/EiC/issues/2006July/Artemisinin.asp. It is widely used for the treatment of malaria, particularly in combination with other drugs such as mefloquine (ASMQ), lumefantrine (such as sold under the trade name Coartem), amodiaquine (such as sold under the trade names Camoquin, Flavoquine, ASAQ), piperaquine (such as sold under the trade name Duo-Cotecxin), Artemisinin and pyronaridine (such as sold under the trade name Pyramax). Artemisinin derivatives are typically used as prodrugs of the biologically active metabolite dihydroartemisinin, which is active during the stage when the parasite is located inside red blood cells. The mechanism through which artemisinin derivatives kill the parasite is believed to act via perturbing redox homeostasis in malaria parasites. Accordingly, such compositions may include free-radical production in the parasite food vacuole and inhibition of a parasite calcium ATPase. A key advantage of artemisinins is rapid action against all of the erythrocytic stages of the parasite, including transmissible gametocytes, resulting in a rapid clinical benefit and decreased transmission of malaria; see Rosenthal, Artesunate for Treatment of Severe Falciparum Malaria, N. Engl J Med 2008, 358:1829-1836.

One of the disadvantages of artemisinin is its poor physical properties resulting in poor bioavailability, limiting its effectiveness. For example, the compound is sparingly soluble in either water or oils and thus not readily absorbable by the gastrointestinal tract. As a result, numerous semi-synthetic derivatives have been developed, including artesunate (water-soluble: for oral, rectal, intramuscular, or intravenous use), artemether (lipid-soluble: for oral, rectal or intramuscular use) dihydroartemisinin, artelinic acid, artenimol and artemotil. As used in the present invention, artemisinin derivatives include, but are not limited to artesunate, dihydroartemisinin, dihydroartemisinin hemisuccinate, dihydrodroartemisinin succinate, sodium artesunate, stabilized forms of artesunate, stabilized forms of sodium artesunate, dihydroartemisitene dimers as described in U.S. Pat. No. 7,098,242, amino-functionalized 1,2,4-trioxanes as described in U.S. Pat. No. 7,071,226, artemisinin endoperoxides as described in U.S. Pat. No. 6,984,640, spiro and dispiro 1,2,4-trioxolane antimalarials as described in U.S. Pat. No. 6,906,205, mixed steroidal 1,2,4,5-tetraoxane compounds as described in U.S. Pat. No. 6,906,098, arteether as described in U.S. Pat. No. 6,750,356, substituted 1,2,4-trioxanes as described in U.S. Pat. No. 6,737,438, Artemisia annua extracts as described in U.S. Pat. No. 6,685,972, artemether as described in U.S. Pat. No. 6,683,193, trioxane derivatives based on artemisinin as described in U.S. Pat. No. 6,649,647, trioxane dimer compounds as described in U.S. RE38,117, conjugates of artelinic acid as described in U.S. Pat. No. 6,461,603, arteethers from dihydroartemisinin as described in U.S. Pat. No. 6,346,631, artemisinine or artemisinene derivatives as described in U.S. Pat. No. 6,306,896, C-10 carbon substituted artemisinin-like trioxane compounds as described in U.S. Pat. No. 6,160,004, water-soluble trioxanes as described in U.S. Pat. No. 6,136,847, alpha arteether as described in U.S. Pat. No. 6,127,405, artemisinin dimers as described in U.S. Pat. No. 5,856,351, (+)-deoxoarteminisinin and analogs of (+)-deoxoartemisinin as described in U.S. Pat. No. 5,225,562, and 10-substituted ether derivatives of dihydroartemisinin as described in U.S. Pat. No. 5,225,427, as well as its salts or other derivatives thereof as known to one of skill in the art.

Artemether is a methyl-ether derivative of dihydroartemisinin derived from artemisinin. It has an IUPAC chemical name of (+)-(3-alpha,5a-beta,6-beta,8a-beta, 9-alpha,12-beta,12aR)-decahydro-10-methoxy-3,6,9-trimethyl-3,12-epoxy-12H-pyrano(4,3-j)-1,2-benzodioxepin, and having a chemical formula:

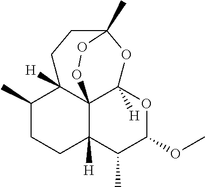

Artemether is known to be effective against blood schizots of several malaria causing parasites, and is used as an ACT drug.

Artesunate, also known as dihydroartemisinin hemisuccinate and its salts, has a IUPAUC chemical name of 3R,5aS,6R,8aS,9R,10S,12R,12aR)-Decahydro-3,6,9-trimethyl-3,12-epoxy-12H-pyrano(4,3-j)-1,2-benzodioxepin-10-ol hydrogen succinate, with a chemical formula of:

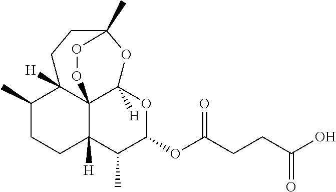

Artesunate is used primarily as a treatment for malaria.

Dosage for Artemisinin, Artemisinin salts and derivatives.

The oral dosage range for Artemisinin, Artemisinin salts and derivatives is between about 1 mg to about 1,500 mg per dose administered one to three times daily. More preferably, the oral dosage range for artemisinin, artemisinin salts and derivatives is between about 20 mg to about 250 mg per dose if taken one to three times daily. Most preferably, the oral dosage ranges for artemisinin, artemisinin salts and derivatives is between about 40 mg to about 100 mg per dose taken one to three times daily.

Berberine, Salts Thereof, and Berberine Derivatives.

Berberine is a quaternary ammonium salt from the protoberberine group of isoquinoline alkaloids. It has a IUPAC name of (5,6-Dihydro-9,10-dimethoxybenzo[g]-1,3-benzodioxolo[5,6-a]quinolizinium), with a chemical formula of:

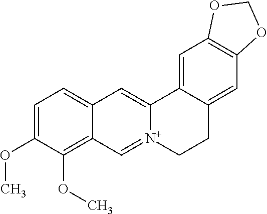 [SMILES: COc1ccc2cc3[n+](cc2c1OC)CCc1cc2c(cc1-3)OCO2]

It is found in various plant species of Berberis (e.g. Berberis aquifolium (Oregon grape), Berberis vulgaris (Barberry), and Berberis aristata (Tree Turmeric)), as well as other plant families, including but not limited to Hydrastis canadensis (Goldenseal), Phellodendron amurense (Amur Cork Tree, Huang Bai, Huang Po, Po Mu) and Coptis chinensis (Chinese Goldthread, Huang-Lian, Huang-Lien), and Tinospora cordifolia, and to a smaller extent in Argemone mexicana (Prickly Poppy) and Eschscholzia californica (Californian Poppy), Rhizoma coptidis Huanglian Jiedu decoction, San-Huang-Xie-Xin-Tang, Xietianwu, Gegen Quinlian, and Shizhu. Berberine is primarily isolated from the roots, rhizomes, stems, and bark. Berberine has been used for medical use in both Ayurvedic and Chinese medicines, and has recently been tested for anti-bacterial activity and use in diarrhea, prostate cancer and diabetes. It is generally considered to be non-toxic and shows no genotoxic activity.

In accordance with the present invention, berberine, berberine derivatives, or its salts may include, but are not limited to, berberine alkaloid, berberine base, berberinehydrochloride, berberine, berberrubine, coreximine, tetrahydropalmatine, jatrorrhizine, 13-hydroxyberberine chloride, coralyne, coralyne chloride, 7,8-dihydro-13-methylberberine, berberine acetone, 13-allylberberine, palmatine, 13-benzylberberine, tetrahydroberberine, tetrahydroprotoberberine 8-cyanodihydroberberine, dimeric protoberberine alkaloids, demethylated protoberberine alkaloids, quataternary protoberberine alkaloids, protoberberineand protoberberine alkaloids, the salts of berberine, including berberine hydrochloride, berberine chloride, berberine sulfate, berberine tannate and other salts known to one of skill in the art.

Dosage Range for Berberine, its Salts and Derivatives.

The oral dosage range of Berberine is from about 50 mg to about 1,500 mg administered in a single dose two to three times daily, not to exceed 4,500 mg per day. More preferably, the dose of berberine is about 100 mg to about 1,000 mg in a single human dose not to exceed 3,000 mg per day, delivered one to three times daily. The most preferable dosage range for a single dose of berberine is about 200 mg to about 500 mg taken one to three times daily.

The present invention is further described by the following non limiting examples. The following examples illustrate embodiments, albeit preferred embodiments, of routes of administration and forms of the composition. While the preferred forms and/or routes are described, other forms, such as but not limited to tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration. The compositions may also be developed for inhalational, oral, rectal, vaginal, parenteral, topical, transdermal, pulmonary, intranasal, buccal, ophthalmic, intrathecal, intravenous or any route of administration.

The dosage form of the present invention may include either immediate or controlled release forms. Each composition can be formulated and manufactured by procedures known to one of skill in the art, and may include 100% composition or a mixture of one or more pharmaceutically acceptable excipients or pharmaceutically acceptable carriers. For use of sublingual sprays, liquid artemether can be prepared as a spray form using, for example, the microfluidization process as described in U.S. Pat. No. 6,861,066.

### Example 1

### Method of Treating an Individual Suffering from a Disease Mediated by a Virus of the Family Flaviviridae

A method of treating an individual suffering from a disease mediated by a virus of the family Flaviviridae, comprises the steps of (1) administering to a mammal, preferably a human, a first composition, the first composition comprising a therapeutically effective amount of an artemisinin derivate, preferably artemether; (2) administering to the mammal a second composition, the second composition comprising a therapeutically effective amount of a second artemisinin derivate, the second artemisinin derivate differing from the first composition, and preferably being artesunate or its pharmaceutically acceptable derivatives at 1 mg to 1,500 mg per dose, preferably about 20 mg to about 250 mg per dose, three times daily, and most preferably about 40 mg to about 100 mg per dose three times daily; and (3) administering to a mammal a third composition, the third composition comprising a therapeutically effective amount of berberine, or its pharmaceutically acceptable derivatives at about 1 mg to about 1,500 mg per dose, particularly about 100 mg to about 1,000 mg per dose three times a day, and more specifically about 200 mg to about 400 mg per dose, two to three times a day.

The following describes an illustrative, albeit preferred delivery treatment schedule:

Day 1: Delivery of Artemether—Single Dose.

Delivery of Artesunate and berberine tablets, dosage according to user age and/or weight every eight hours.

Day 2: Delivery of Artesunate and berberine tablets, dosage according to user age and/or weight every eight hours.

Day 3: Delivery of Artesunate and berberine tablets, dosage according to user age and/or weight every eight hours.

### Example 2

### Treatment for Adults Over 165 Pounds (75 Kg) Suffering from a Viral Infection Using Sublingual Delivery of an Artemether Solution: Dengue Fever

A method of treating an individual suffering from Dengue Fever comprises the steps of (1) administering to an adult mammal, preferably a human, a first composition, the first composition comprising a therapeutically effective amount of artemether; (2) administering to the adult mammal a second composition, the second composition comprising a therapeutically effective amount of an artesunate or its pharmaceutically acceptable derivatives; and (3) administering to the adult mammal a third composition, the third composition comprising a therapeutically effective amount of berberine, or its pharmaceutically acceptable derivatives.

An adult human, over 165 pounds (75 Kg), begins the method in accordance with the present invention by administering the first loading dose of the artemether. The artemether is delivered to the individual using a sublingual spray bottle containing 60 mg of an artemether solution per bottle. Use of a spray offers advantages over injectable form by providing a method of delivery that is easy to administer, does not require a trained medical professional such as a doctor or nurse, and avoids the problems associated with needles, such as fear of spreading disease such as HIV or Hepatitis C and proper disposal. In addition, sublingual drug delivery, particularly sprays, allows the active ingredients improved absorption and enhanced bioavailability.

In use, the user simply removes the bottle from the transportation/delivery packaging and displaces any safety features, such as any tamper proofing outside wrapping, secured to the bottle. After removing the seal from the spray bottle, the adult individual lifts his/her tongue and pumps the spray bottle with artemether solution into the mouth so the solution is delivered to the area under the tongue. The bottle remains in that position until the contents of the bottle are fully delivered to the area under the tongue. The liquid is preferably held under the tongue for a predetermined time period, preferably 30 seconds. Any liquid remaining in the user's mouth is then swallowed. After waiting for a predetermined time period, such as 1 minute to 1 hour, and more preferably 30 minutes, the user administers the second composition comprising a dosage of artesunate, such as 2×50 mg tablets, 3 doses per day (300 mg per day), or every eight hours for 2 days. The user administers the third composition comprising a dosage of berberine, such as 2×400 mg tablets, 3 doses per day (2,400 mg), or every eight hours. Alternatively, the second and third compositions can be administered simultaneously with the first composition.

### Example 3

### Treatment for Adult Humans 66 (30 Kg)-165 Pounds (75 Kg) Suffering from a Viral Infection Using Sublingual Delivery of an Artemether Solution: Dengue Fever

An adult human weighing 66 (30 kg)-165 pounds (75 Kg) begins the method in accordance with the present invention by administering the first loading dose of the artemether. The artemether is delivered to the individual using a sublingual spray bottle containing 60 mg artemether solution per bottle. The user simply removes the bottle from the transportation/delivery packaging and displaces any safety features, such as outside wrapping to indicate tampering, secured to the bottle. After removing the seal from the spray bottle, the adult individual lifts his/her tongue and pumps the spray bottle with artemether solution into the mouth so the solution is delivered to the area under the tongue. The bottle remains in that position until the contents of the bottle are fully delivered to the area under the tongue. The liquid is preferably held under the tongue for a predetermined time period, preferably 30 seconds. Any liquid remaining in the user's mouth is then swallowed. After waiting for a predetermined time period, such as 1 minute to 1 hour, and more preferably 30 minutes, the user administers the second composition comprising a dosage of artesunate, such as 1×50 mg tablets, 3 doses per day (150 mg), or every eight hours for 2 days. The user administers the third composition comprising a dosage of berberine, such as 1×400 mg tablet, 3 doses per day (1200 mg), or every eight hours. Alternatively, the second and third compositions can be administered simultaneously with the first composition.

### Example 4

### Treatment for Human a Child 33 Pounds (15 kg)-66 Pounds (30 Kg) Suffering a Viral Infection Using Sublingual Delivery of an Artemether Solution: Dengue Fever

For children or individuals in the range of between 33 pounds (15 kg)-66 pounds (30 Kg), the treatment is the same as described in Example 3.

### Example 5

### Treatment for Infants and Toddlers 33 Pounds Up to 3 Years Old (Under 15 Kg) Suffering Viral Infection Using Sublingual Delivery of an Artemether Solution: Dengue Fever

For infants and toddlers 33 pounds up to 3 years old (under 15 kg), the treatment is similar to Example 1. The dosage delivered to the user includes artemether delivered to the individual using a sublingual spray containing 60 mg artemether solution per bottle, 25 mg artesunate per dosage, 3 doses per day (75 mg total per day), or every eight hours for 2 days, and 200 mg berberine per dosage, 3 doses per day (600 mg total per day), or every eight hours. For infants and toddlers, the tablets can be ground up and mixed with liquids that allow delivery into the users, such as milk, juice, and syrups.

### Example 6

### Treatment for Individuals Suffering Viral Infection Using Injectable Delivery of Artemether Solution: Dengue Fever

A method of treating an individual suffering from Dengue Fever comprises the steps of (1) administering to a mammal, preferably a human, a first composition, the first composition comprising a therapeutically effective amount of artemether; (2) administering to the mammal a second composition, the second composition comprising a therapeutically effective amount of artesunate or its pharmaceutically acceptable derivatives; and (3) administering to the mammal a third composition, the third composition comprising a therapeutically effective amount of berberine, or its pharmaceutically acceptable derivatives.

In a preferred embodiment, the method of treatment can be facilitated by the use of a kit comprising an injectable artemether solution as a single dosage unit contained in an ampoule, injection vial, or as an individual single, preloaded injection syringe with needle, a first packet containing artesunate tablets, and a second packet containing berberine tablets. An individual user begins the method in accordance with the present invention by administering the first loading dose of the artemether. The artemether is delivered to the individual as an injection, via IV or IM mode of administration. The second and third compositions are delivered as previously described in Examples 2-4, depending on the weight and age of the individual user.

As described in the above examples, the components of the present invention can be supplied in a kit to aid in delivery and use of the method in accordance with the present for treatments in areas where distribution channels and/or medical communities are not easily accessible or established. In accordance with the present invention, the kit may include a secure delivery package in the form of a self sealing blister pack, zip lock packs, standup pouches, foil pouches which include the single use spray bottle and one day, two day, three day, four day, five plus day packs of the artesunate and berberine. If injectable artemether is used, needles, syringes, injection bottles, or pre-loaded syringes with capped needles may be included. The kit preferably contains directions and/or any other instructions for the proper use and storage of the components of the kit.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of treating an individual suffering from a disease mediated by an arthropod comprising the steps of:
administering to an individual a first composition, said first composition comprising a therapeutically effective amount of an artemisinin derivate;
administering to said individual a second composition, said second composition comprising a therapeutically effective amount of a second artemisinin derivate, said second artemisinin derivate differing from said first composition;
administering to said individual a third composition comprising an effective amount of berberine, or its pharmaceutically acceptable derivatives.

2. The method of treating an individual suffering from a disease mediated by an arthropod according to claim 1 wherein said arthropod is a mosquito.

3. The method of treating an individual suffering from a disease mediated by an arthropod according to claim 1 wherein said first composition is administered sublingually.

4. The method of treating an individual suffering from a disease mediated by an arthropod according to claim 3 wherein said sublingual delivery is via a spray.

5. The method of treating an individual suffering from a disease mediated by an arthropod according to claim 1 wherein said first composition is administered to said individual via an injection.

6. The method of treating an individual suffering from a disease mediated by an arthropod according to claim 1 wherein said second and third compositions are delivered via an oral route.

7. The method of treating an individual suffering from a disease mediated by an arthropod according to claim 6 wherein second and third compositions are in the form of a pill or capsule.

8. The method of treating an individual suffering from a disease mediated by an arthropod according to claim 6 further including the steps of administering additional doses of said second and third compositions to said individual for a predetermined time period.

9. The method of treating an individual suffering from a disease mediated by an arthropod according to claim 1 wherein said individual has Dengue Fever.

10. A method of treating an individual suffering from a disease mediated by a virus of the family Flaviviridae comprising the steps of:
administering to an individual a first composition, said first composition comprising a therapeutically effective amount of an artemisinin derivate;
administering to said individual a second composition, said second composition comprising a therapeutically effective amount of a second artemisinin derivate, said second artemisinin derivate differing from said first composition;
administering to said individual a third composition, said third composition comprising an effective amount of berberine, or its pharmaceutically acceptable derivatives.

11. The method of treating an individual suffering from a disease mediated by a virus of the family Flaviviridae according to claim 10 wherein said virus is responsible for Dengue Fever, Japanese encephalitis, Kyasanur Forest disease, Murray Valley encephalitis, St. Louis encephalitis, Tick-borne encephalitis, West Nile encephalitis, Yellow fever, and Hepatitis C Virus Infection.

12. The method of treating an individual suffering from a disease mediated by a virus of the family Flaviviridae according to claim 11 wherein said first composition is artemether.

13. The method of treating an individual suffering from a disease mediated by a virus of the family Flaviviridae according to claim 12 wherein said artemether is administered sublingually.

14. The method of treating an individual suffering from a disease mediated by a virus of the family Flaviviridae according to claim 10 wherein said second composition is artesunate.

15. A method of treating an individual suffering from Dengue Fever comprising the steps of:
administering to an individual a first composition, said first composition comprising a therapeutically effective amount of artemether or its pharmaceutically acceptable salt;
administering to said individual a second composition, said second composition comprising a therapeutically effective amount of artesunate or its pharmaceutically acceptable salt;
administering to an individual a third composition, said third composition comprising a therapeutically effective amount of berberine, or its pharmaceutically acceptable salt.

16. The method of treating an individual suffering from Dengue Fever to according to claim 15 wherein said first composition is administered sublingually.

17. The method of treating an individual suffering from Dengue Fever according to claim 16 wherein said sublingual delivery is via a spray.

18. The method of treating an individual suffering from Dengue Fever according to claim 15 wherein said first composition is administered to said individual via an injection.

19. The method of treating an individual suffering from Dengue Fever according to claim 16 wherein said second and said third compositions are delivered via an oral route.

20. The method of treating an individual suffering from Dengue Fever according to claim 16 wherein said second and said third compositions are in the form of a pill or capsule.

21. The method of treating an individual suffering from Dengue Fever according to claim 16 wherein said step of administering said artemether includes delivering a single dosage.

22. The method of treating an individual suffering from Dengue Fever according to claim 16 further including the steps of administering additional doses of said second and said third compositions for a predetermined time period.

23. A kit for treating a microbial disease comprising:
a first composition, said first composition comprising a therapeutically effective amount of an artemisinin derivate;
a second composition, said second composition comprising a therapeutically effective amount of a second artemisinin derivate, said second artemisinin derivate differing from said first composition;
a third composition comprising an effective amount of berberine, or its pharmaceutically acceptable derivatives.

24. The kit for treating a microbial disease according to claim 23 wherein said first composition is artemether, said second composition is artesunate, and said third composition is berberine.

25. The kit for treating microbial disease according to claim 24 wherein said artemether is a spray.

26. The kit for treating a microbial disease according to claim 24 wherein said artemether is in an injectable form.

27. The kit for treating a microbial disease according to claim 26 wherein said artemether is preloaded to a syringe.

28. The kit for treating a microbial disease according to claim 24 wherein said second and said third compositions are in pre-packed dosages.
